(19) United States
(12) Patent Application Publication
(10) Pub. No.: US 2004/0146953 A1
(43) Pub. Date: Jul. 29, 2004

(54) Assay

(76) Inventors: Walter Philip Blackstock, Stevenage (GB); Richard Stephen Hale, Stevenage (GB); Rabinder Prinjha, Harlow (GB); Adele Rowley, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/466,391

(22) PCT Filed: Jan. 18, 2002
(86) PCT No.: PCT/GB02/00214

(30) Foreign Application Priority Data
Jan. 18, 2001 (GB) 0101313.5

(51) Int. Cl. G01N 33/574

(52) U.S. Cl. 435/7.23

(57) ABSTRACT

A method of identifying a modulator BACE function, the method comprising: (i) providing (a) a BACE polypeptide; (b) a Nogo polypeptide; (c) a test agentunder conditions that would permit binding of a BACE polypeptide (a) to a Nogo polypeptide (b) in the absence of the test agent (c) wherein said BACE polypeptide (a) is BACE or a variant thereof or a fragment of either thereof capable of binding Nogo; and polypeptide (b) is Nogo or a variant thereof or a fragment of either thereof capable of binding BACE; (i) monitoring BACE mediated activity; and (ii) determining thereby whether the test agent is a modulator of BACE activity. Modulators identified by a method of the invention and use of such modulators in the manufacture of a medicament for the treatment of disorders responsive to the modulation of BACE activity such as Alzheimer's disease.

### FIELD OF THE INVENTION

This invention relates to methods of identifying modulators of BACE and/or Nogo activity and their use in the treatment of conditions in which abnormal activity of BACE is implicated such as Alzheimer's disease.

### BACKGROUND TO THE INVENTION

Cleavage of amyloid precursor protein (APP) by β-secretase (and an additional activity known as γ-secretase) results in the generation of short Aβ fragments that are a major component of amyloid plaques found in the brains of Alzheimer's sufferers. Substantial evidence links amyloid deposition to the pathogenesis of Alzheimer's disease. APP cleavage, on the lumenal side of the membrane, by β-secretase is though to be rate limiting for Aβ generation in vivo.

An aspartyl protease called BACE (also known as Asp2 or memapsin2) has recently been shown to be responsible for β-secretase activity. BACE is a type I transmembrane protein with a large lumenal domain containing the protease domain.

BACE may have other substrates in addition to APP but none have yet been identified. Recombinant BACE clearly cleaves peptides based on the APP cleavage site in vitro and also cleaves APP when BACE and APP are co-expressed in vivo. As expected, BACE exhibits greater activity towards APP_{swedish}. Recombinant BACE may however have broad specificity and it is difficult to identify additional substrates purely from sequence information.

At least three Nogo isoforms are generated by alternative splicing of transcripts derived form the NogoA gene. The C-terminal third of all three isoforms shares high homology (approximately 70% at the amino acid level) with the reticulon protein family. NogoA, the largest isoform, has been shown to inhibit axon regeneration in culture. It is thought that the normal role of Nogo proteins is to prevent axon sprouting in the uninjured central nervous system. NogoA is localised to central nervous system myelin and is highly expressed in oligodendrocytes; NogoB and NogoC are expressed in some neurons and several non-neural tissues. All Nogo isoforms surprisingly have a C-terminal ER-retention motif but at least some of NogoA protein is thought to reach the cell surface. All 3 Nogo isoforms have 2 potential trans-membrane domains. Both the C and N termini may be cytoplasmically exposed and a 66 amino acid loop separated by the TM domains may be located extracellularly.

### SUMMARY OF THE INVENTION

The present inventors have identified a novel interaction between BACE and Nogo. The interaction between BACE and Nogo provides a new therapeutic intervention point in disorders which are responsive to modulation of BACE function, and more specifically in Alzheimer's disease. In addition Nogo is now proposed as a target for identifying agents which may be useful in the treatment of Alzheimer's disease.

Accordingly the invention provides a method of identifying a modulator BACE function, the method comprising:

(i) providing

(a) a BACE polypeptide

(b) a Nogo polypeptide

(c) a test agent

under conditions that would permit binding of a BACE polypeptide (a) to a Nogo polypeptide (b) in the absence of the test agent (c) and wherein said BACE polypeptide (a) is BACE or a variant thereof or a fragment of either thereof capable of binding Nogo and said Nogo polypeptide (b) is Nogo or a variant thereof or a fragment of either thereof capable of binding BACE.

(ii) monitoring BACE mediated activity; and

(iii) determining thereby whether the test agent is a modulator of BACE activity.

In a further aspect, the invention provides A method for identification of a modulator of Nogo activity, which method comprises:

(i) contacting Nogo or a variant thereof or a fragment of either thereof which maintains a Nogo function with a test agent; and

(ii) monitoring for Nogo activity

thereby determining whether the test agent is a modulator of Nogo activity.

The invention also provides:

a modulator identifiable by a method according to the invention;

use of a modulator identifiable by a method according to the invention in the manufacture of a medicament for the treatment or prophylaxis of Alzheimer's disease;

use of a Nogo polypeptide or a polynucleotide encoding a Nogo polypeptide in the manufacture of a medicament for the treatment, prophylaxis or diagnosis of Alzheimer's disease wherein said Nogo polypeptide is Nogo or a variant thereof or fragment of either thereof which is capable of binding BACE;

a method of treatment of Alzheimer's disease comprising administering an effective amount of a Nogo polypeptide, a polynucleotide encoding a Nogo polypeptide or a modulator identified by a method of the invention to a human or animal in need of such treatment wherein said Nogo polypeptide is Nogo or a variant thereof or fragment of either thereof which is capable of binding BACE; and

a method of treatment of Alzheimer's disease comprising:

(i) identifying a modulator of Nogo activity; and

(ii) administering a therapeutically effective amount of the said modulator to a patient in need thereof.

### BRIEF DESCRIPTION OF THE FIGURES

FIG. 1A

Tas10 (mouse #4) sections stained with Nogo-A monoclonal antibody (6D5). Note presence of a ring of staining surrounding plaques of all sizes.

FIG. 1B

Left panels show sections stained with an Anti-Nogo-A polyclonal antibody at two different magnifications. The two panels on the right show double staining using the same Nogo-A polyclonal and an anti-Abeta monoclonal 1E8.

FIG. 1C

Similar ring-like staining with Asp2/BACE specific monoclonal antibody (9B21) in these Tas10 transgenic mice.

FIG. 1D

Asp2/BACE staining of Tas10 transgenic mouse brain sections with monoclonal 9B21 shows a distinctive cellular staining extending in processes into the amyloid plaque core.

FIG. 1E

Sequential sections from mouse #3 (18 month old Tas10 transgenic) stained for Nogo-A (left panel) and Asp2/BACE (right panel). Note the lower levels of Nogo-A overexpression in animals with a lower amyloid load (compared with animal 4 in previous sections). Both proteins show a similar overlapping distribution in all plaques present in both sections).

FIG. 2

Embryonic hippocampal neurons express Nogo-A throughout the cytoplasm, with some apparent surface localisation. Concentrations of Nogo-A immunoreactivity are seen in varicosities or synaptic structures along the processes.

FIG. 3A

Transient transfections produce high levels of Asp2/BACE and Nogo-A protein in SHSY5Y-APPswe cells. The left panel shows cells transfected with Asp2/BACE and stained with Anti-myc tag clone 9E10. The right panel shows cells transfected with Nogo-A and stained with a specific monoclonal antibody (clone 6D5).

FIG. 3B

Cotransfection of Asp2/BACE-myc and Nogo-A into SHSY5Y cells. Panels showing the following stains. Top left Hoescht nuclear stain; Top right Asp2/BACE-myc; Bottom left Nogo-A polyclonal 67; Bottom right Merge of Asp2/BACE and Nogo-A stains. Note codistribution of a pool of Asp2/BACE with Nogo-A in the double-transfected cells.

FIG. 3C

Cotransfection of Asp2/BACE-myc and Nogo-B into SHSY5Y cells. Panels showing the following stains. Top left Hoescht nuclear stain; Top right Asp2/BACE-myc; Bottom left Nogo-B polyclonal 66; Bottom right Merge of Asp2/BACE and Nogo-B stains. Note codistribution of a pool of Asp2/BACE with Nogo-B in the double-transfected cells.

FIG. 4A

Nogo Isoform Overexpression Does Not Enhance Abeta Production in SHSH5Y-APPswe Cells. Left panel shows effects of transfections into SHSY5Y-APPswedish cells on Abeta X40 production. The right panel shows the effects of transfections on AbetaX-42 production. Bars represent the activity after transfection with GFP, Asp2/BACE, Nogo-A and Nogo-B respectively in each panel. Note Asp2/BACE increases Abeta production in these cells at this cell density while single transfection of the two Nogo isoforms appears not to enhance production at this cell density.

FIG. 4B.

Co-expression of Nogo isoforms with Asp2/BACE modulates Ab production in SHSY5Y-APPswe cells. Assays measuring AbetaX40 (left panel) and AbetaX-42 (right panel) were conducted on cells cotransfected with the indicated combinations of DNA in the following order. Asp2/BACE plus GFP; Asp2/BACE plus Nogo-A; Asp2/BACE plus Nogo-B; Asp2/BACE plus Nogo-C. Double transfections can be used to assay the activity of Nogo isoforms on APP processing.

### BRIEF DESCRIPTION OF THE SEQUENCES

SEQ ID No: 1 shows the BACE nucleotide coding sequence and amino acid sequence.

SEQ ID No: 2 is the amino acid sequence of BACE.

SEQ ID No: 3 shows the NogoB nucleotide coding sequence and amino acid sequence.

SEQ ID No: 4 is the amino acid sequence of NogoB.

SEQ ID No: 5 is an amino acid fragment of NogoB identified in an assay to identify proteins which bind BACE.

SEQ ID No: 6 is an amino acid fragment of NogoB identified in an assay to identify proteins which bind BACE.

SEQ ID No: 7 is an amino acid fragment of NogoB identified in an assay to identify proteins which bind BACE.

SEQ ID No: 8 shows the NogoA nucleotide coding sequence and amino acid sequence.

SEQ ID No: 9 is the amino acid sequence of NogoA.

SEQ ID No: 10 shows the NogoC nucleotide coding sequence and amino acid sequence.

SEQ ID No: 11 is the amino acid sequence of NogoC.

SEQ ID No: 12 shows the BACE2 nucleotide coding sequence and amino acid sequence.

SEQ ID No: 13 is the amino acid sequence of BACE2.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The invention provides a method for identifying a modulator of BACE activity and a method for identifying a modulator of Nogo activity. A modulator may modulate the interaction between BACE and Nogo.

A BACE polypeptide for use in accordance with the may comprise a naturally occurring BACE such as BACE (accession numbers AF190725, P56817) having the amino acid sequence of SED ID No: 1 or SEQ ID No: 2 or may comprise a variant or fragment of BACE which may be a naturally occurring BACE such as BACE2 (accession numbers AF204944, Q9Y5ZO) having the amino acid sequence of SEQ ID No: 12 or 13 or another unidentified isoform or splice variant which is homologous to or retains the desired function of a known BACE. Such a variant or fragment of BACE for use in the invention is one which is capable of binding to a Nogo polypeptide having the sequence of SEQ ID No: 5, 6 or 7. Preferably a variant or fragment of BACE is capable of binding to full length NogoA, NogoB and/or NogoC. A preferred variant or fragment of BACE may also comprise an aspartyl protease active site. Such preferred variants or fragments retain the ability to cleave proteins comprising a β-secretase cleavage site. Preferably such variants and fragments encompass amino acid residues 93 to 96 (DTGS) and/or residues 289-292 (DSGT) of SEQ ID No: 2 or residues 109-112 (DTGS) and/or 300-303 (DSGT) of SEQ ID No: 13.

A Nogo polypeptide for use in accordance with the invention is one which capable of binding BACE. The Nogo polypeptide may be Nogo A (accession No. AJ251383), Nogo B (accession No. AB015639) or Nogo C (accession No. AF125103). The Nogo polypeptide comprises the amino acid sequence of SEQ ID No: 4, 9 or 11 or a functional variant or functional fragment thereof. Preferably, the Nogo polypeptide comprises the amino acid sequence of SEQ ID No: 4 or a functional variant or a functional fragment thereof. A variant may comprise a naturally occurring isoform or splice variant. A variant or fragment of SEQ ID No: 4, 9 or 11 for use in accordance with the invention is capable of binding to BACE. Particularly preferred variants of Nogo comprise other members of the reticulon family. Particularly preferred fragments and variants of SEQ ID No: 4 comprise the amino acid sequence shown in SEQ ID No: 5, 6 and 7.

To determine whether a variant or fragment of BACE is capable of binding to Nogo the variant or fragment can be contacted with Nogo under conditions suitable for the formation of a complex between BACE and Nogo. Similarly, to determine whether a variant or fragment of Nogo is capable of binding to BACE, the variant or fragment can be contacted with BACE under conditions suitable for the formation of a complex between Nogo and BACE. Any one of the assays described herein can be carried out in the absence of a test agent to determine the binding capabilities of these proteins.

Proteins with naturally occurring amino acid sequences are preferred for use in the assays. Preferred proteins are human proteins but homologues from other mammalian species, or other animal species may be used. Any allelic variant or species homologue of the defined proteins may be used. References to a variant or fragment of the protein as described below relates to a variant or fragment of both BACE and Nogo. For all the proteins described herein for use in an assay of the invention, the ability of the variant or fragment to bind Nogo or BACE as appropriate is preferably maintained.

Polypeptides that have been artificially mutated but retain Nogo or BACE binding activity or other BACE or Nogo activity may also be used in the invention. Such mutants may be generated by techniques well known in the art, including site directed mutagenesis, random mutagenesis and restriction enzyme digestion and ligation. A protein for use in the invention preferably has more than about 65% sequence identity to a natural protein, more preferably at least 70%, at least 80%, at least 90%, at least 95%, at least 97% or at least 99% sequence identity thereto over a region of at least 20, preferably at least 30, for instance at least 40, at least 60 or at least 100 contiguous amino acids or over the full length of SEQ ID No: 2 or SEQ ID No: 4. Amino acid substitutions may be made, for example from 1, 2 or 3 to 10, 20 or 30 substitutions. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |

| | |
|---|---|
| AROMATIC | H F W Y |

The entire protein sequence of each of the proteins used in the assay may be present. Fragments of the proteins and variants described above that retain the ability to bind to the second component in the binding assay, i.e. BACE for Nogo polypeptides and Nogo for BACE polypeptides may also be used in the invention. Alternatively variants or fragments of Nogo which retain a function of Nogo may be used in assays to identify modulators of Nogo activity. Preferred fragments of SEQ ID No: 2 will be at least 30, e.g. at least 100, at least 200, at least 300, at least 400 or at least 450 amino acids in length. Preferred fragments of SEQ ID No: 4 will be at least 30, e.g. at least 100, at least 200 or at least 250 amino acids in length. A fragment may comprise part of a polypeptide, for example a chimeric polypeptide. A chimeric protein may be used to facilitate the purification of a BACE or Nogo polypeptide. For example, the lumenal domain of BACE (amino acids 1 to 460 of SEQ ID No: 2) may be fused to human IgG at the carboxy-teminus.

As used herein, a BACE polypeptide (a) is used to refer to BACE having the sequence of SEQ ID No: 2 or a variant thereof or a fragment of either thereof which variant or fragment is capable of binding to Nogo, or to a variant or a fragment of Nogo which is capable of binding to BACE.

As used herein, a Nogo polypeptide (a) is used to refer to Nogo having the sequence of SEQ ID No: 4 or a variant thereof or a fragment of either thereof which variant or fragment is capable of binding to BACE, or to a variant or a fragment of BACE which is capable of binding to Nogo.

The polypeptides for use in the invention may be chemically modified, e.g. post-translationally modified. For example, they may be glycosylated or comprise modified amino acid residues. The polypeptides may be tagged to aid detection or purification, for example using a HA, histidine, T7, myc or flag tag. The BACE polypeptide (a) and the Nogo polypeptide (t)) may be tagged with different labels which may assist in identification of a BACE/Nogo complex.

Assays

Any suitable assay format may be used for identifying a modulator of a BACE activity, for example a modulator of a BACE/Nogo interaction.

As the first step of the method for identifying a modulator of BACE function, (a) a BACE polypeptide comprising the sequence of SEQ ID No: 2 or a variant thereof or fragment of either sequence capable of binding to Nogo; (b) a Nogo polypeptide comprising the sequence of SEQ ID No: 4 or a variant thereof or a fragment of either sequence capable of binding to BACE; and (c) a test agent are contacted under conditions that would permit binding of (a) to (b) in the absence of a test agent. An activity of BACE is then monitored. For example, the interaction between the BACE polypeptide (a) and the Nogo polypeptide (b) may be analysed. The interaction between the BACE polypeptide (a) and the Nogo polypeptide (b) in the presence of a test agent may be compared with the interaction between the BACE polypeptide (a) and the Nogo polypeptide (b) in the absence of the test agent to determine whether the test agent modulates the binding of BACE polypeptide (a) and the Nogo polypeptide (b) and thereby whether the test agent enhances or inhibits the binding BACE to Nogo.

The test agent can be contacted with a cell harbouring a polynucleotide or expression vector encoding a BACE polypeptide (a) and a polynucleotide or expression vector encoding a Nogo polypeptide (b). Optionally the cell may harbour a polynucleotide or expression vector encoding a test agent, wherein the test agent is a peptide. The cell typically allows transcription and translation of the polynucleotides or vectors so that the polypeptides are expressed in the same cell.

The test agent may be provided in the extracellular medium used for washing, incubating or growing the cell. The test agent may modulate the interaction of the Nogo polypeptide (b) with the BACE polypeptide (a) indirectly from outside the cell, for example by interacting with an extracellular domain of BACE or Nogo or may be taken up into the cell from the extracellular medium. Where the BACE polypeptide (a) and the Nogo polypeptide (b) are coexpressed in a cell, the cell may express both proteins naturally, for example the cell may be a neuronal cell grown in a primary culture, or the cell may express both proteins recombinantly, or the cell may naturally express one protein and be transformed to express the other protein recombinantly.

The cell may be transiently or stably transfected or transformed. The BACE polypeptide (a) and the Nogo polypeptide (b) may both be transiently expressed, both stably expressed or one may be stably expressed and the other transiently expressed. Cells can be transfected by methods well known in the art, for example, by electroporation, calcium phosphate precipitation, lipofection or heat shock. The proteins may be expressed in mammalian cells such as human cells or non-mammalian cells such as yeast or bacteria. It is preferred that the cells are in culture. Preferred cell lines which may be used include HEK293, COS and PC12 cells.

A cell expressing a BACE polypeptide (a) or a cell homogenate, a cell lysate, a membrane preparation or a protein preparation derived from a cell expressing a BACE polypeptide (a) can be contacted with a cell expressing a Nogo polypeptide (b) or a cell homogenate, a cell lysate, a membrane preparation or a protein preparation derived from cells expressing a Nogo polypeptide (b).

The conditions which permit binding of a BACE polypeptide (a) to a Nogo polypeptide (b) in an extracellular environment can be determined by carrying out the assay in the absence of a test agent.

A control assay in which the agent to be tested is omitted and an assay in which a test agent is included can be carried out in parallel or subsequently. The results of the experiments using the test agent and the control experiments can be used to determine whether the test agent inhibits or enhances binding.

The agent tested may be tested with any other known interacting protein combinations to exclude the possibility that the test agent is a general inhibitor of protein/protein interactions.

Where the BACE polypeptide (a) used in the assay is a variant or fragment of SEQ ID No: 2, or the Nogo polypeptide (b) used in the assay is a variant or fragment of SEQ ID No: 4, the assay is preferably run first in the absence of a test agent to ensure that the variant or fragment exhibits the activity being monitored, such as binding activity or protease activity.

A number of biochemical and molecular cell biology protocols known in the art can be used to analyse the interaction of a BACE polypeptide (a) and a Nogo polypeptide (b) (see for example Sambrook et al., 1989). Some specific examples are outlined below:

The BACE/Nogo interaction can be determined directly using a binding assay. For example, a radiolabelled BACE polypeptide (a) may be incubated with a Nogo polypeptide (b) in the presence and absence of the test agent and the effect of the test agent on the binding of the BACE polypeptide (a) to the Nogo polypeptide (b) is monitored. Typically, the radiolabelled BACE polypeptide (a) is incubated with cell membranes containing the Nogo polypeptide (b) until equilibrium is reached. The membranes can then be separated from non-bound radiolabelled BACE polypeptide (a) and dissolved in scintillation fluid to allow the radioactive content to be determined by scintillation counting. Non-specific binding of the agent may also be determined by repeating the experiment in the presence of a saturating concentration of a non-radioactive BACE polypeptide (a). Preferably a binding curve is constructed by repeating the experiment with various concentrations of the radiolabelled BACE polypeptide, both in the presence and absence of the test agent.

A yeast-2 hybrid assay system may be used to monitor the effect of a test agent on the BACE/Nogo interaction. For example, a polynucleotide encoding a BACE polypeptide (a) can be cloned into GALA binding domain vector (GAL4_{BD}) and a Nogo polynucleotide (b) can be cloned into a GAL4 activation domain fusion vector (GAL4_{AD}). The GAL4_{AD} and GAL4_{BD} vectors can then be expressed in yeast and the resulting β-galactosidase activity in the presence and absence of the test agent can be assayed and quantified using the substrate o-nitrophenol ∃-D-galactopyranoside (ONPG) using a liquid nitrogen freeze fracture regime as described by Harshman et al., 1998.

A “pull-down” assay system may also be used. Isolated BACE polypeptide (a) may be immobilised on a surface and binding of a Nogo polypeptide (b) to the surface may be monitored in the presence and absence of the test agent. The assay can also be carried out by immobilizing a Nogo polypeptide (b) and measuring the binding of a BACE polypeptide (a) to the immobilized protein.

Alternatively, of a BACE polypeptide (a) may be immunoprecipitated, immunopurified or affinity purified from a cell extract of cells co-expressing a BACE polypeptide (a) and a Nogo polypeptide (b). Coprecipitating/copurifying Nogo polypeptide (b) can then be detected, for example using Western blotting techniques or by radiolabelling recombinantly expressed proteins, and quantified using a phosphorimager or scintillation counter. The test agent is generally added to the cells or the cell growth medium prior to preparation of the cell lysate.

The assays may also be carried out monitoring other BACE functions. For example, the step of monitoring BACE activity may involve assessment of BACE protease activity or the effect of binding of BACE to other proteins. The assay may typically involve determination of APP processing. For example, cleavage of a peptide comprising the β-secretase cleavage site (SEVKM/DAEFR or SEVNL/DAEFR) may be monitored. Alternatively, any suitable method may be used to monitor protease activity. Suitable methods are described in Vassar et al. (1999) Science 286, 735-741, Hussain et al. (1999) Molecular and Cellular Neuroscience 14, 419-427, Sinha et al. (1999) Nature 402, 537-540 or Yan et al. (1999) Nature 402, 533-537.

An important aspect of the present invention is the use of a Nogo polypeptide (b) in screening methods to identify compounds that may act as modulators of Nogo activity and in particular compounds that may be useful in treating BACE associated disease. Any suitable form may be used for the assay to identify a modulator of Nogo activity. In general terms, such screening methods may involve contacting Nogo polypeptide (b) with a test agent and then measuring activity. For example, the neurite inhibitory activity of Nogo may be monitored. Neurite inhibitory activity may be monitored using any suitable assay format such as a dorsal root ganlion (DRG) neurite outgrowth assay, a DRG growth cone collapse assay, a neuronal cell line (for example, PC12 cell) neurite outgrowth assay or a fibroblast (such as NIH 3T3) cell spreading assay.

Modulator activity can be determined by contacting cells expressing a Nogo polypeptide (b) of the invention with an agent under investigation and monitoring the effect of the modulator on Nogo activity. The cells expressing the polypeptide may be in vitro or in vivo. The polypeptide of the invention may be naturally or recombinantly expressed. Preferably, the assay is carried out in vitro using cells expressing recombinant polypeptide.

Candidate Modulators

A modulator of BACE or Nogo function may exert its effect by binding directly to BACE or Nogo or may have an upstream effect which prevents the BACE/Nogo interaction occurring or which inhibits BACE or Nogo mediated activity.

A modulator may directly inhibit the interaction of BACE with Nogo or inhibit interaction between Nogo and a ligand. A candidate modulator (test agent) may comprise a fragment of a Nogo which is capable of binding BACE or a Nogo ligand but which lacks any functional activity. Alternatively, a candidate modulator may comprise a fragment of BACE which is capable of binding Nogo but which lacks any functional activity.

Antibodies, or antibody fragments that specifically bind to BACE or Nogo or chemical compounds capable of binding these proteins are also candidate compounds. An antibody, or other compound, “specifically binds” to a protein when it binds with high affinity to the protein for which it is specific but does not bind or binds with only low affinity to other proteins. A variety of protocols for competitive binding or immunoradiometric assays to determine the specific binding capability of an antibody are well known in the art (see for example Maddox et al. 1993). Such immunoassays typically involve the formation of complexes between the “specific protein” and its antibody and the measurement of complex formation.

Furthermore, combinatorial libraries, defined chemical identities, peptide and peptide mimetics, oligonucleotides and natural product libraries, such as display libraries (e.g. phage display libraries) may also be tested. The candidate agents may be chemical compounds. Batches of the candidate agents may be used in an initial screen of, for example, ten agents per reaction, and the agents of batches which show inhibition tested individually.

Modulators

A modulator of BACE activity is an agent which produces a measurable reduction or increase in binding of a Nogo polypeptide (b) to BACE polypeptide (a) in the assays described above, or an effect on BACE activity or Nogo activity.

Preferred inhibitors are those which inhibit binding by at least 10%, at least 20%, at least 30%, at least 40% at least 50%, at least 60%, at least 70%, at least 80%, at least 90%, at least 95% or at least 99% at a concentration of the inhibitor of 1 μg ml⁻¹, 10 μg ml⁻¹, 100 μg ml⁻¹, 500 μg ml⁻¹, 1 mg ml⁻¹, 10 mg ml⁻¹ or 100 mg ml⁻¹.

Preferred activators are those which activate binding by at least 10%, at least 25%, at least 50%, at least 100%, at least, 200%, at least 500% or at least 1000% at a concentration of the activator 1 μg ml⁻¹, 10 μg ml⁻¹, 100 μg ml⁻¹, 500 μg ml⁻¹, 1 mg ml⁻¹, 10 mg ml⁻¹ or 100 mg ml⁻¹.

The percentage inhibition or activation represents the percentage decrease or increase in expression/activity in a comparison of assays in the presence and absence of the test agent. Any combination of the above mentioned degrees of percentage inhibition or activation and concentration of inhibitor or activator may be used to define an inhibitor or activator of the invention, with greater inhibition or activation at lower concentrations being preferred.

Test agents which show activity in assays such as those described above can be tested in in vivo systems, an animal model. Candidate inhibitors could be tested for their ability to decrease BACE mediated signalling, for example by with APP processing.

Candidate activators could be tested for their ability to increase BACE mediated signalling. Ultimately such agents would be tested in animal models of the target disease states.

Therapeutic Use

Modulators of the interaction between BACE and Nogo or of BACE activity or of Nogo activity identified by the methods of the invention may be used for the treatment or prophylaxis of a disorder that is responsive to modulation of BACE activity or Nogo activity.

In particular, neuronal disorders such as cognitive disorders including Alzheimer's disease may be treated. A modulator of BACE or Nogo activity may be used to alleviate the symptoms or to improve the condition of a patient suffering from such a disorder.

Modulators of BACE or Nogo activity may be useful in enhancing cognitive function. This may be useful in treating neurodegenerative diseases such as Alzheimer's disease or in enhancing cognitive function following injury to the brain.

Nogo polypeptides and polynucleotides encoding Nogo polypeptides may also be used in the treatment or prophylaxis of such disorders.

The invention therefore provides a use of a polynucleotide which encodes Nogo or a variant thereof which is capable of binding BACE or a fragment of either thereof which is capable of binding BACE, which polynucleotide comprises:

(a) the sequence of SEQ ID No: 3, 8 or 10; or

(b) a sequence that hybridizes to the complement of SEQ ID No: 3, 8 or 10; or

(c) a sequence that is degenerate as a result of the genetic code with respect to a sequence defined in (a) or (b); or

(d) a sequence that is complementary to a polynucleotide defined in (a), (b) or (c);

in the manufacture of a medicament for use in a method of treatment of a disorder that is responsive to modulation of BACE activity.

A polynucleotide comprising a sequence that hybridizes to the complement of the coding sequence of SEQ ID No: 3, 8 or 10 can hydridize at a level significantly above background. Background hybridization may occur, for example, because of other cDNAs present in a cDNA library. The signal level generated by the interaction between a polynucleotide of the invention and the complement of the coding sequence of SEQ ID No: 3, 8 or 10 is typically at least 10 fold, preferably at least 100 fold, as intense as interactions between other polynucleotides and the coding sequence of SEQ ID No: 3, 8 or 10. The intensity of interaction may be measured, for example, by radiolabelling the probe, e.g. with ³²P. Selective hybridisation may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.03M sodium chloride and 0.003M sodium citrate at about 60° C.). However, such hybridization may be carried out under any suitable conditions known in the art (see Sambrook et al., 1989). For example, if high stringency is required, suitable conditions include 0.2×SSC at 60° C. If lower stringency is required, suitable conditions include 2×SSC at 60° C.

A nucleotide sequence which is capable of selectively hybridizing to the complement of the DNA coding sequence of SEQ ID No: 3, 8 or 10 will generally have at least 60%, at least 70%, at least 80%, at least 90%, at least 95%, at least 98% or at least 99% sequence identity to the coding sequence of SEQ ID No: 3 over a region of at least 20, preferably at least 30, for instance at least 40, at least 60, more preferably at least 100 contiguous nucleotides or most preferably over the full length of SEQ ID No: 3. Methods of measuring nucleic acid and protein homology are well known in the art. For example the UWGCG Package provides the BESTFIT program which can be used to calculate homology (Devereux et al., 1984). Similarly, the PILEUP and BLAST algorithms can be used to line up sequences (for example as described in Altschul, 1993 and Altschul et al., 1990.) Many different settings are possible for such programs. According to the invention, the default settings may be used.

Any combination of the above mentioned degrees of sequence identity and minimum sizes may be used to define polynucleotides encoding a Nogo polypeptide, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The coding sequence of SEQ ID No: 3, 8 or 10 may be modified by nucleotide substitutions, for example from 1, 2 or 3 to 10, 25, 50 or 100 substitutions. The polynucleotide of SEQ ID No: 4, 9 or 11 may alternatively or additionally be modified by one or more insertions and/or deletions and/or by an extension at either or both ends. The modified polynucleotide generally encodes a protein that can bind BACE. Typically the protein encoded by the modified polypeptide has neurite inhibitory activity. Degenerate substitutions may be made and/or substitutions may be made which would result in a conservative amino acid substitution when the modified sequence is translated, for example as shown in the Table above.

Polynucleotides may comprise DNA or RNA. They may also be polynucleotides which include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or lifespan of polynucleotides of the invention.

Polynucleotides encoding a Nogo polypeptide may be produced recombinantly, synthetically, or by any means available to those of skill in the art. They may also be cloned by standard techniques. The polynucleotides are typically provided in isolated and/or purified form.

Although in general the techniques mentioned herein are well known in the art, reference may be made in particular to Sambrook et al, 1989, Molecular Cloning: a laboratory manual.

A polynucleotide may also be an essential component in an assay of the invention, a probe (or template for designing a probe) for identifying proteins that may be used in the invention or a test agent. The nucleotides may be involved in recombinant protein synthesis as well as therapeutic agents in their own right, utilised in gene therapy techniques. Antisense sequences, may also be used in gene therapy, such as in strategies for down regulation of expression of Nogo.

Polynucleotides for use in the invention can be inserted into expression vectors. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al.

Polynucleotides may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of a disorder responsive to modulation of BACE activity, in particular for the treatment neurodegenerative disorders such as Alzheimer's disease.

Examples of suitable viral vectors include herpes simplex viral vectors and retroviruses, including lentiviruses, adenoviruses, adeno-associated viruses and HPV viruses (such as HPV-16 or HPV-18). Gene transfer techniques using these viruses are known to those skilled in the art. Retrovirus vectors for example may be used to stably integrate the polynucleotide giving rise to the antisense RNA into the host genome. Replication-defective adenovirus vectors by contrast remain episomal and therefore allow transient expression.

Another aspect of the present invention is the use of polynucleotides encoding the Nogo polypeptides of the invention to identify mutations in Nogo genes which may be implicated in human disorders. Identification of such mutations may be used to assist in diagnosis of or susceptibility to Alzheimer's or other conditions associated with BACE and in assessing the physiology of such disorders. Polynucleotides may also be used in hybridisation studies to monitor for expression of Nogo genes and in particular for up or down regulation of Nogo expression.

The formulation of an agent for use in preventing or treating any of the above mentioned conditions will depend upon factors such as the nature of the agent identified, whether a pharmaceutical or veterinary use is intended, etc. Typically a modulator is formulated for use with a pharmaceutically acceptable carrier or diluent. For example it may be formulated for topical, parenteral, intravenous, intramuscular, subcutaneous, intraocular, transdermal or oral administration. A physician will be able to determine the required route of administration for each particular patient. The pharmaceutical carrier or diluent may be, for example, an isotonic solution.

The dose of an agent may be determined according to various parameters, especially according to the agent used; the age, weight and condition of the patient to be treated; the route of administration; and the required regimen. Again, a physician will be able to determine the required route of administration and dosage for any particular patient.

Modulators may have to be administered to specific sites, or otherwise targeted to brain cells. For example, the modulator may be delivered to neurons. This may be achieved, for example, by delivery via a viral strain such as herpes simplex virus. Viral vectors comprising polynucleotides of the invention are described above. The viral vector delivery method may be used in the case of administration of, for example, polynucleotides of the invention. The vector may further comprise a promoter or other regulatory sequence that is specific to certain neurons.

The polynucleotides and vectors of the invention may be administered directly as a naked nucleic acid construct. Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™).

Typically, nucleic acid constructs are mixed with the transfection agent to produce a composition. Preferably the naked nucleic acid construct, viral vector comprising the polynucleotide or composition is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, or transdermal administration.

The pharmaceutical composition is administered in such a way that the polynucleotide of the invention, viral vector for gene therapy, can be incorporated into cells at an appropriate area. When the polynucleotide of the invention is delivered to cells by a viral vector, the amount of virus administered is in the range of from 10⁶ to 10¹⁰ pfu, preferably from 10⁷ to 10⁹ pfu, more preferably about 10⁸ pfu for adenoviral vectors. When injected, typically 1-2 ml of virus in a pharmaceutically acceptable suitable carrier or diluent is administered. When the polynucleotide of the invention is administered as a naked nucleic acid, the amount of nucleic acid administered is typically in the range of from 1 μg to 10 mg.

Where the polynucleotide giving rise to the product is under the control of an inducible promoter, it may only be necessary to induce gene expression for the duration of the treatment. Once the condition has been treated, the inducer is removed and expression of the polypeptide of the invention ceases. This will clearly have clinical advantages. Such a system may, for example, involve administering the antibiotic tetracycline, to activate gene expression via its effect on the tet repressor/VP16 fusion protein.

The use of tissue-specific promoters will be of assistance in the treatment of disease using the polypeptides, polynucleotide and vectors of the invention. It will be advantageous to be able express therapeutic genes in only the relevant affected cell types, especially where such genes are toxic when expressed in other cell types.

The routes of administration and dosages described above are intended only as a guide since a skilled physician will be able to determine readily the optimum route of administration and dosage for any particular patient and condition.

The following Examples illustrates the invention.

### EXAMPLE 1

The target protein BACE1 was amplified by PCR using primers:

AGGAAGTGGAAGTGGCCACCATGGCCCAAGCCCTGCCC and GTAGGGGTAATTGGCCTTCAGCAGGGAGATGTCATC.

The PCR product was cloned into an expression vector such that an 8 residue histidine tag was inserted in frame at the C-terminus. This construct was transfected into HEK293 cells that were expanded under conditions selecting for the expression construct.

In a representative experiment BACE was affinity purified from 65 mg of the membrane-enriched fraction derived from approximately 10⁸ transfected cells. Membrane proteins, solubilized in 1% CHAPSO, were incubated with 500 μl Ni-NTA resin and eluted with 150 mM imidazole. The resulting eluate was precleared with sepharose (Pierce) for 1 hour. After discarding the sepharose, the eluate was incubated with anti-His antibody (Serotec) covalently bound to sepharose (Pierce aminolink plus) at 4° C. overnight. Finally immunoprecipitated proteins were resuspended in sample buffer, separated on a 4-12% bis-tris gel under reduced conditions and stained with colloidal Coomassie blue. Bands specific to the tagged cell line were excised and in-gel digested with trypsin. All trypsin digested peptides were subjected to LC/MS/MS and proteins were identified by searching a non-redundant protein database.

Proteins identified in a representative experiment are summarised below.

NogoB (ASY) peptides identified:

| | | | |
|---|---|---|---|
| 1 | MEDLDQSPLV | SSSDSPPRPQ | PAFKYQFVRE PEDEEEEEEE |
| | EEEDEDEDLE | | |
| 51 | ELEVLERKPA | AGLSAAPVPT | APAAGAPLMD FGNDFVPPAP |
| | RGPLPAAPPV | | |
| 101 | APERQPCWDP | SPVSSTVPAP | SPLSAAAVSP SKLPQDDEPP |
| | ARPPPPPPAS | | |
| 151 | VSPQAEPVWT | PPAPAPAAPP | STPAAPKRRG SSGSVVVDLL |
| | YWRDIKKTGV | | |
| 201 | VFGASLFLLL | SLTVFSIVSV | TAYIALALLS VTISFRIYKG |
| | VIQAIQKSDE | | |
| 251 | GHPFRAYLES | EVAISEELVQ | KYSNSALGHV NCTIKELRRL |
| | FLVDDLVDSL | | |
| 301 | KFAVLMWVFT | YVGALFNGLT | LLILALISLF SVPVIYERHQ |
| | AQIDHYLGLA | | |
| 351 | NKNVKDAMAK | IQAKIPGLKR | KAE |

The underlined peptide is sufficient to distinguish NogoB from other Nogo isoforms.

### EXAMPLE 2

### Differentating PC/2 Cells and Measuring Neurite Outgrowth.

PC12 cells are plated at 1×10⁵ cells per well in 1.5 ml complete DMEM using a 6-well plate. Plated cells are incubated overnight at 37° C. in 5% CO₂ to allow them to attach. Cells are washed with complete, prewarmed DMEM. 1.5 ml low serum DMEM+/−NGF+/−Bace+/−Nogo+/−test agent is added to each well. A range of concentrations of Bace, Nogo and NGF of 0, 0.1, 1, 10, 50 and 100 ng/ml are tested. The test agent is added to differnet wells at concentrations of 0, 0.001, 0.01, 0.1, 1 and 10 nM. The cells are then incubated for 48 hours at 37° C. in 5% CO₂ to allow them to attach.

After 48 Hours the cells should be about 50% confluent and are fixed in 4% paraformaldehyde phosphate for measurement of neurite outgrowth. Acid fucin stain is diluted (2×) in PBS, mixed and filter sterilised through a syringe. 500 μl of diluted stain is added to each well. Cells are incubated in the stain for 2 minutes at room temperature and washed in PBS (1 ml) four times. Fuchin stains cell bodies and neurites red. Neurite number and length/cell body are measured on an inverted microscope using appropriate image analysis and software.

### EXAMPLE 3

Sections from adult transgenic mice (designated Tas10) overexpressing human APP constructs containing the Swedish mutation were stained with a variety of antibody markers. These were chosen to allow the detection of amyloid plaques and proteins associated with or around them.

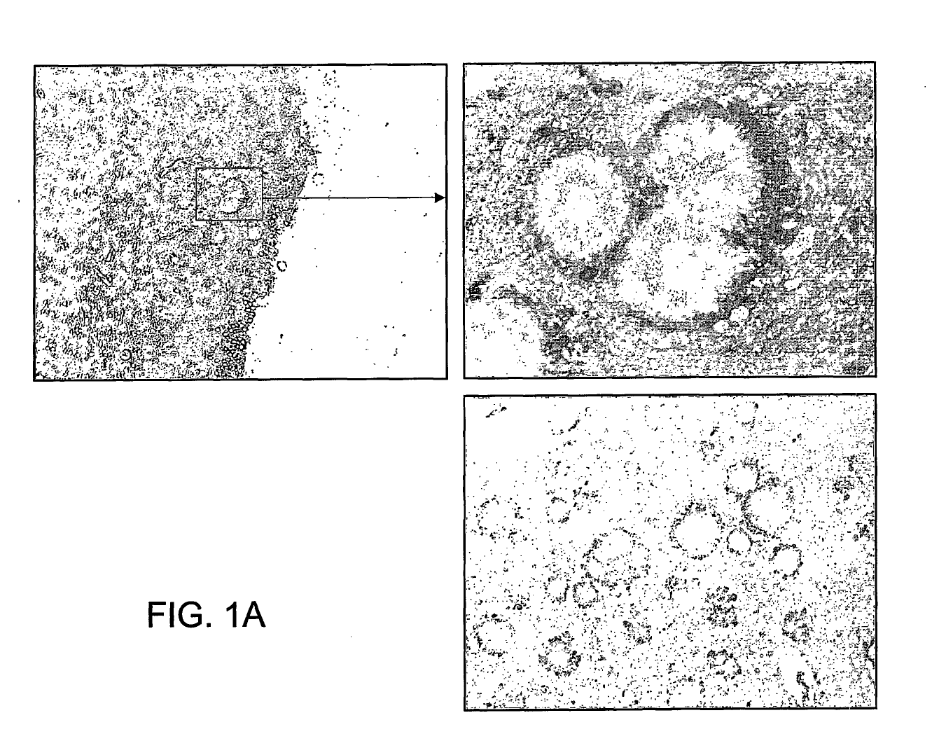

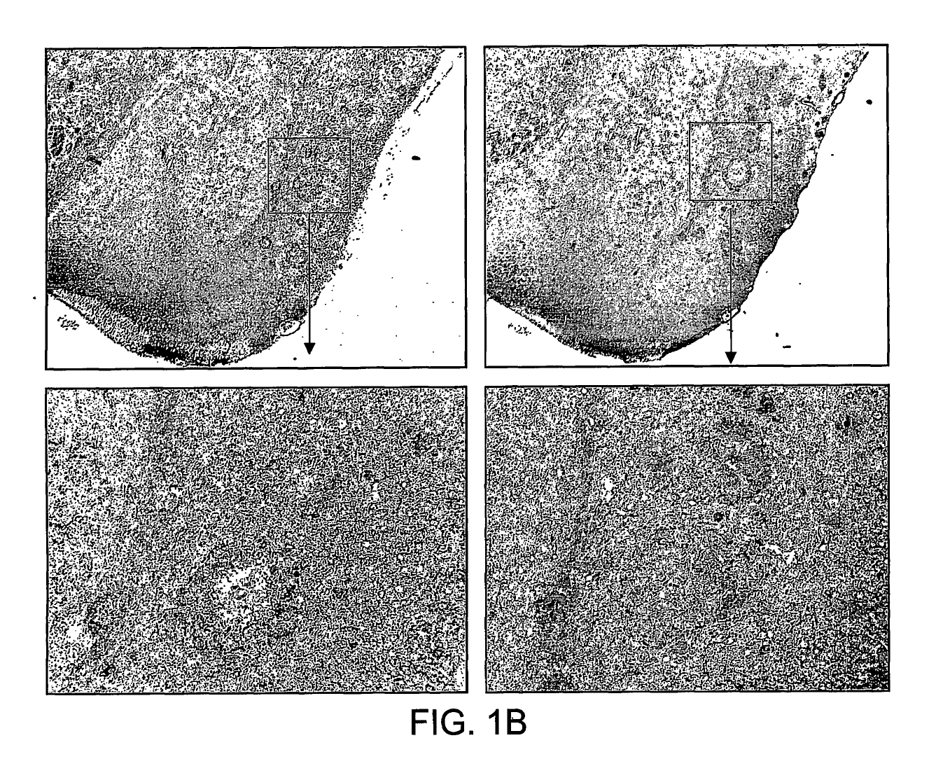

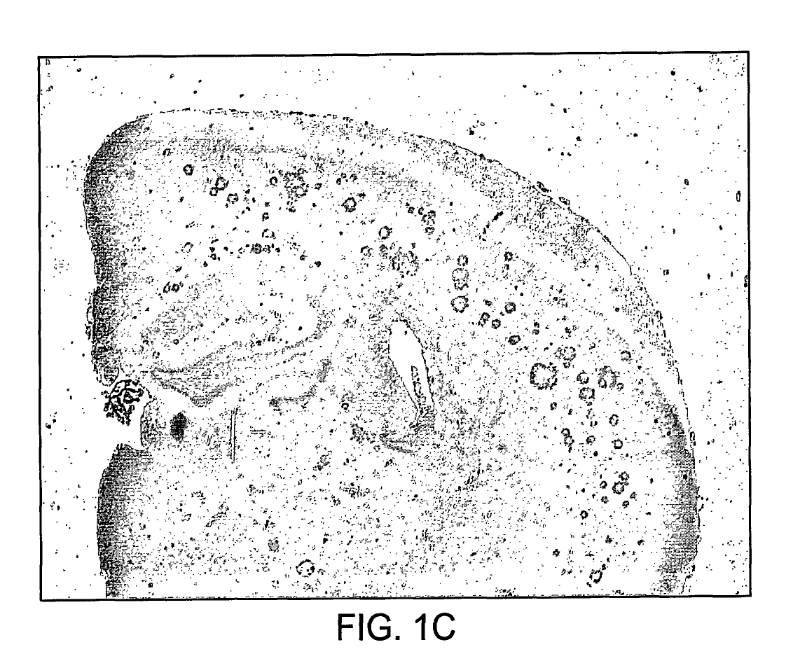

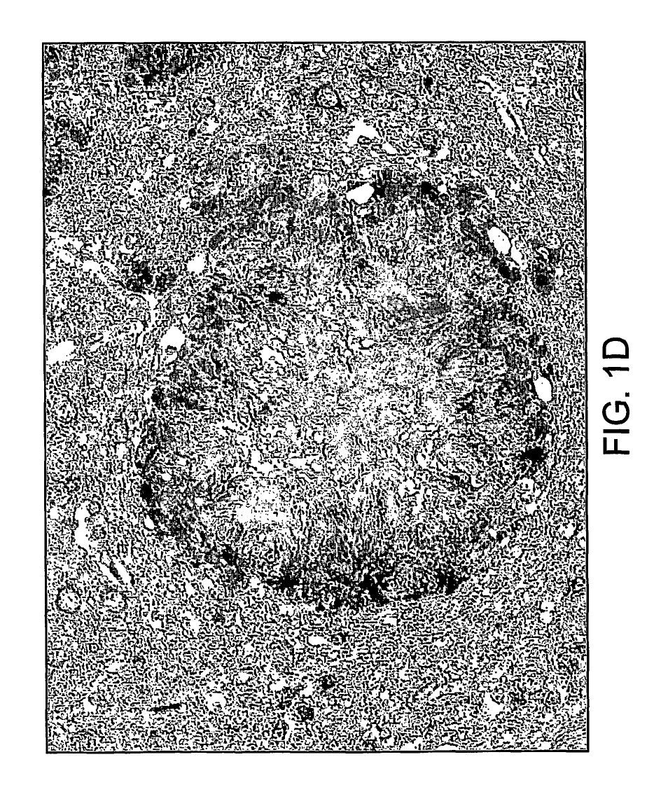

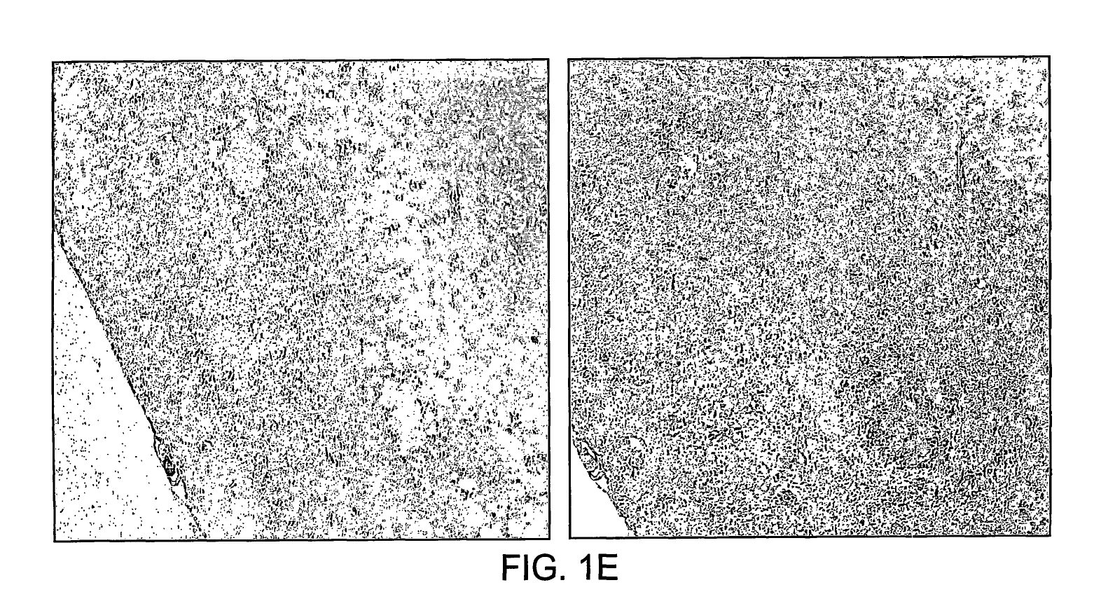

In a representative experiment a monoclonal antibody against the Nogo-A protein (clone 6D5) was found to stain the sections (for example Tas10 animal 4) in a pattern reminiscent of a ring surrounding most if not all plaque like structures (FIG. 1A).

In order to confirm that this was actually plaque associated staining, further sections were double-stained with a monoclonal antibody (clone 1E8) directed against the amyloid peptide together with an affinity purified polyclonal antibody directed against Nogo-A (Alpha Diagnostics). In these sections an intense pink coloured product was deposited in the plaques labelling amyloid protein and these were surrounded by a darker blue/purple product corresponding to sites of Nogo-A protein expression. These observations confirmed our earlier finding of overexpression of Nogo-A around the edges of plaques in regions of new amyloid deposition (FIG. 1B).

A number of additional antibody markers including neurofilament (Chemicon), GFAP (Sigma) and phospho-tau (clone AT8) all failed to produce a pattern similar to that seen with the Nogo-A antibodies. A monoclonal antibody directed against Asp2/BACE (clone 9B21) was used to stain sections from the same animal and were found to produce a ring like staining pattern most closely resembling the Nogo-A staining (FIG. 1C). In subsequent serial sections and double-staining experiments it was apparent that the Nogo-A and Asp2/BACE proteins show a remarkably close association FIG. 1E). The only distinction that could be observed was that the Nogo-A stain was more diffusely distributed than the Asp2/BACE staining which appeared to label discrete cellular structures with features characteristic of neurons in this region (FIG. 1D).

Further confirmation of the correlation of elevated Nogo-A expression with progressive pathology was found in the staining pattern of sections from transgenic animals displaying a lower plaque load. In these sections fewer and smaller plaques were seen and these were surrounded by less intense Nogo-A staining (FIG. 1E).

### EXAMPLE 4

To determine the source of the Nogo-A found in the deposits surrounding the plaques cultured neurons that are known to be sensitive to amyloid peptide induced toxicity (in this case embryonic hippocampal neurons) were studied. Similar results can be obtained with other types of cultured neurons.

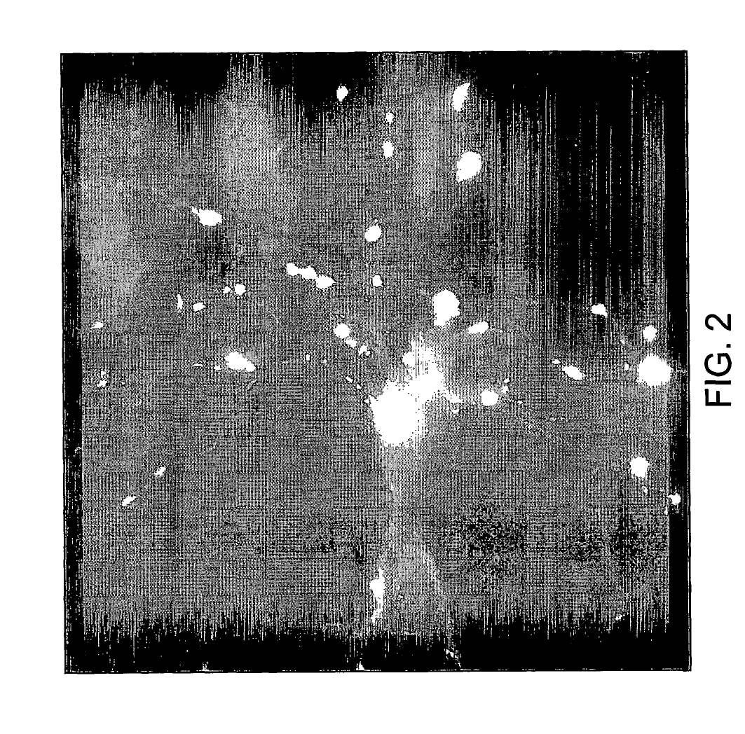

Cultured hippocampal neurons were fixed and stained with a Nogo-A specific monoclonal; the cells displayed prominent cytoplasmic and axonal staining with some apparent cell surface clusters. In relatively mature cultures the Nogo-A can be seen to also show some concentration in varicosities along the processes that may represent synapses (FIG. 2). Treatment of such cultures with Abeta1-42 peptide can be used to determine their effects on Nogo-A expression and further used as a system to identify molecules that Alter those responses as a means of modifying the progression and pathology of diseases such as (but not limited to) Alzheimer's disease.

### EXAMPLE 5

Human neuroblastoma cells (SHSY5Y) stably over-expressing the human APP gene caring the Swedish mutation which predisposes affected individuals to early onset Alzheimer's disease can provide a useful system in which to study the effects of modifying agents on processing of APP into toxic Abeta peptide fragments. These cells normally secrete readily detectable levels of Abeta peptide into the culture medium. Transfection of Nogo-A, Nogo-B or Nogo-C alone or in combination with BACE/Asp2 can be used to determine the effects of overexpression of these proteins on APP. Repression of Abeta formation by Nogo isoforms would be likely to indicate that the overexpression of Nogo-A seen around the plaques in the Tas10 mice represents a compensatory/protective mechanism while an increase would suggest that elevated expression contributes to the pathology of Alzheimer's disease in particular and neurodegenerative diseases in general. Screens of molecules altering the interactions of Nogo with BACE/Asp2 could thus be configured to identify agonists or antagonists of the binding as required.

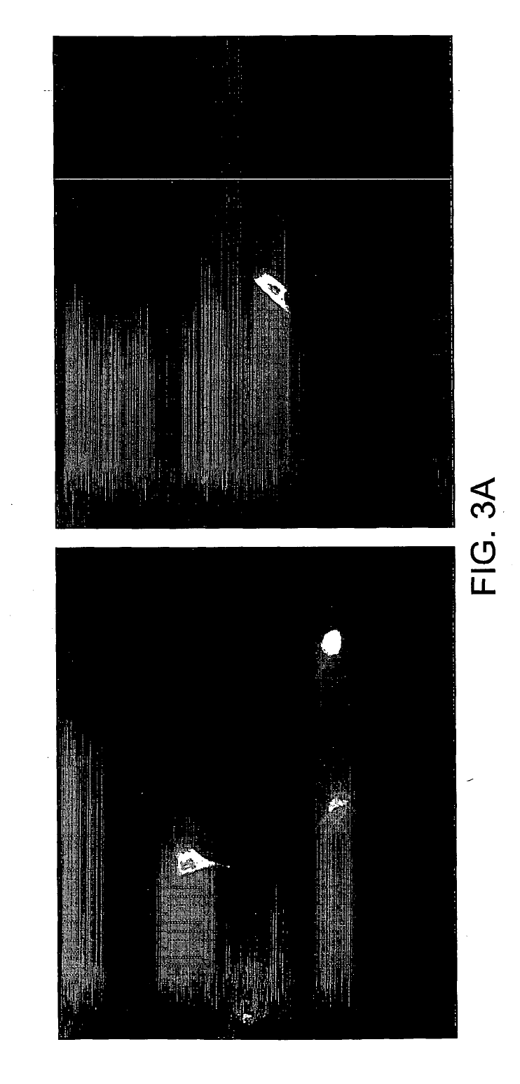

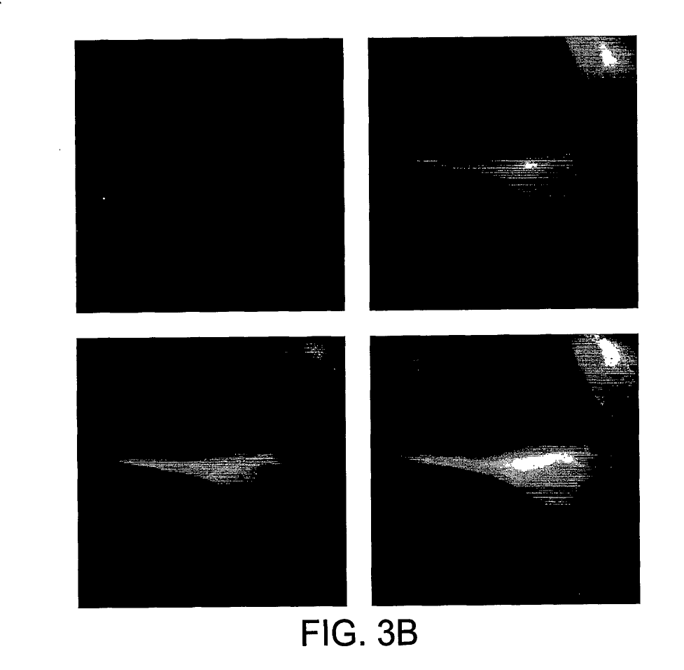

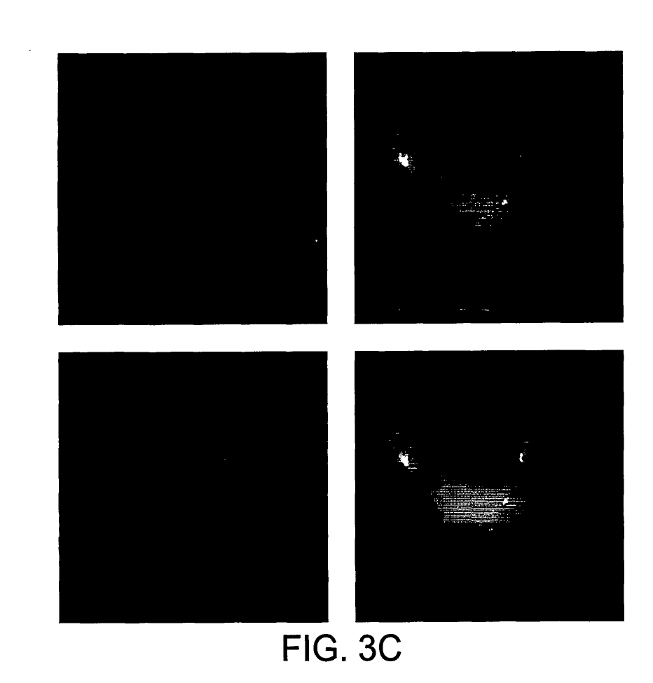

cDNAs encoding Nogo isoforms were transfected into cultured SHSY5Y-APPswe cells and expressed proteins detected in fixed cells with immunohistochemistry. Asp2 was detected using a c-terminal myc epitope (FIG. 3A left panel) while the Nogo-A (FIG. 3A right panel) and B isoforms were detected using isoform specific rabbit polyclonal antibodies (designated 67 and 66 respectively or monoclonal Anti-Nogo-A 6D5 as indicated in the figure legends). These experiments confirm that it is possible to overexpress these proteins in this cellular background and can be used as the basis of an assay for modulators of Nogo influenced APP processing. Furthermore, in double transfected cells, microscopic examination was able to demonstrate the co-localisation of some of the transfected proteins within the cells. These data suggest that both Nogo-A (FIG. 3A) and Nogo-B (FIG. 3B) show the ability to interact with Asp2/BACE in an appropriate cellular background and that this interaction may well be responsible for altering the processing of APP during plaque formation.

### EXAMPLE 6

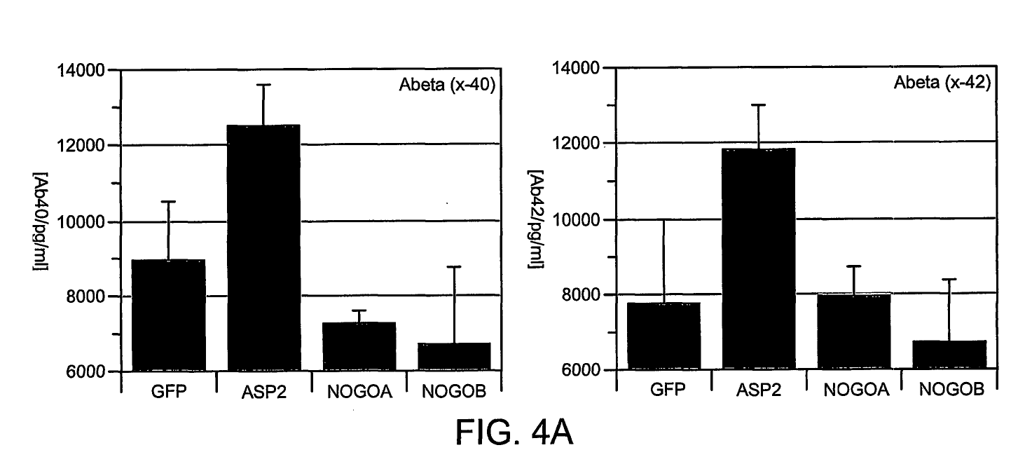

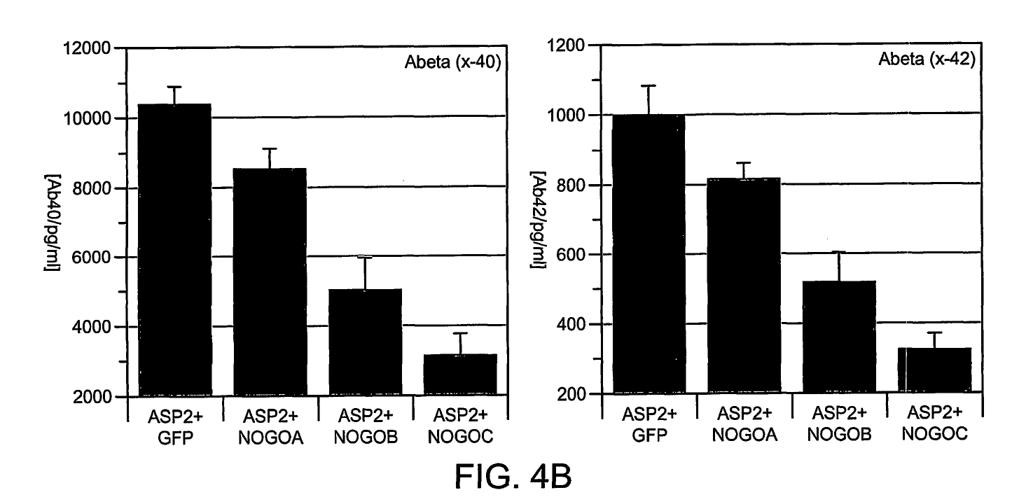

The human neuroblastoma cell-line SHSY5Y-APPswedish can be transfected with cDNAs encoding test constructs and the effects on APP processing and secretion of Abeta into the media can be measured using ELISA assays. In experiments performed at low cell-densities we were able to detect an enhanced production of Abeta x40 and x42 following transfection of an Asp2/BACE construct (FIG. 4A). Cells transfected with Nogo-A or Nogo-B expressing constructs in parallel were found not to produce the same increase in Abeta production suggesting they are not sufficient alone for enhanced amyloidogenesis.

In further experiments the effects of co-expression of all three Nogo isoforms with Asp2/BACE was tested. Analysis of conditioned media suggests that the Nogo isoforms appear to significantly reduce the levels of detectable Abeta peptide (FIG. 4B). The observed alterations in Abeta levels may be due to the direct intracellular interaction of Asp2/BACE with Nogo which was found by co-immunoprecipitation of these proteins from cell lysates. This is supported by their co-localisation in transfected cells and therefore the modulation may be at the level of sub-cellular localisation. Since all the Nogo isoforms have a C-terminal ER retention motif the observed changes in Asp2/BACE activity with increased Nogo expression could cause the retention of more Asp2/BACE within the endoplasmic reticulum away from the sites of normal APP processing.

1. A method of identifying a modulator of BACE function, the method comprising:
(i) providing
(a) a BACE polypeptide;
(b) a Nogo polypeptide;
(c) a test agent
under conditions that would permit binding of a BACE polypeptide (a) to a Nogo polypeptide (b) in the absence of the test agent (c) wherein said BACE polypeptide (a) is BACE or a variant thereof or a fragment of either thereof capable of binding Nogo; and polypeptide (b) is Nogo or a variant thereof or a fragment of either thereof capable of binding BACE;
(ii) monitoring BACE mediated activity; and
(iii) determining thereby whether the test agent is a modulator of BACE activity.

2. A method according to claim 1 wherein step (ii) comprises monitoring the interaction between (a) and (b).

3. A method according to claim 2 wherein the modulator inhibits the binding of (a) to (b).

4. A method according to claim 2 wherein the modulator enhances the binding of (a) to (b).

5. A method according to claim 1 wherein step (ii) comprises monitoring protease activity.

6. A method according to claim 5 wherein said protease activity is β-secretase cleavage of amyloid precursor protein.

7. A method according to claim 5 or 6 wherein the modulator inhibits said protease activity.

8. A method according to any one of the preceding claims wherein the BACE polypeptide (a) is BACE or a said fragment thereof.

9. A method according to any one of the preceding claims wherein the Nogo polypeptide (b) is NogoB or a said fragment thereof.

10. A method for identification of a modulator of Nogo activity, which method comprises:
(i) contacting a Nogo polypeptide or a variant thereof or a fragment of either thereof which maintains a Nogo function with a test agent and
(ii) monitoring for Nogo activity
thereby determining whether the test agent is a modulator of Nogo activity.

11. A method according to claim 10 wherein the Nogo activity comprises the ability of the polypeptide to interact with BACE, or a variant thereof or a fragment of either thereof.

12. A modulator of BACE activity which is identifiable by a method according to any one of claims 1 to 9.

13. A modulator of Nogo activity which is identifiable by a method according to claim 10 or 11.

14. Use of a modulator according to claim 12 or 13 in the manufacture of a medicament for the treatment or prophylaxis of Alzheimer's disease.

15. Use of a Nogo polypeptide, or a polynucleotide encoding a Nogo polypeptide in the manufacture of a medicament for the treatment, prophylaxis or diagnosis of Alzheimer's disease wherein said Nogo polypeptide is Nogo or a variant thereof or fragment of either thereof which is capable of binding BACE.

16. A method of treatment of Alzheimer's disease, which method comprises administering an effective amount of a Nogo polypeptide, a polynucleotide encoding a Nogo polypeptide or a modulator according to claim 12 or 13 to a human or animal in need of such treatment wherein said Nogo polypeptide is Nogo or a variant thereof or fragment of either thereof which is capable of binding BACE.

17. A method of treatment of Alzheimer's disease, which method comprises:
(i) identifying a modulator of Nogo activity; and
administering a therapeutically effective amount of the said modulator to a patient in need thereof
